SEQUENCE LISTING (3 sequences)

SEQ ID NO: 1 - protein, 1466 aa, Homo sapiens
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 2 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPAGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKTSRLPIIDVAPLDVGAPDQEFGFDVGPVCFL

SEQ ID NO: 3 - protein, 1366 aa, Homo sapiens
MLSFVDTRTLLLLAVTLCLATCQSLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPNGSAGPPGFPGAPGPKGEIGAVGNAGPTGPAGPRGEVGLPGLSGPVGPPGNPGANGLTGAKGAAGLPGVAGAPGLPGPRGIPGPPGAAGTTGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPVGARGEPGNIGFPGPKGPTGDPGKNGDKGHAGLAGARGAPGPDGNNGAQGPPGPQGVQGGKGEQGPAGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAHGAVGAPGPAGATGDRGEAGAAGPAGPAGPRGSPGERGEVGPAGPNGFAGPAGAAGQPGAKGERGGKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGFKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMEAIKVYCDFPTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADHEFFVDIGPVCFK

(19) United States
(12) Patent Application Publication
(10) Pub. No.: US 2006/0100138 A1
(43) Pub. Date: May 11, 2006

(54) Implantable collagen compositions

(76) Inventors: David R. Olsen, Menlo Park, CA (US); James W. Polarek, Sausalito, CA (US); Chunlin Yang, Bellemead, NJ (US)

Correspondence Address: FIBROGEN, INC.;INTELLECTUAL PROPERTY DEPARTMENT, 225 GATEWAY BOULEVARD, SOUTH SAN FRANCISCO, CA, 94080, US

(21) Appl. No.: 10/985,570

(22) Filed: Nov. 10, 2004

(51) Int. Cl. C07K 14/78

(52) U.S. Cl. 514/8; 623/23.72; 530/356

(57) ABSTRACT

The present invention relates to collagen compositions suitable for use in various tissue augmentation procedures.

### FIELD OF THE INVENTION

The present invention relates to collagen compositions suitable for use in various tissue augmentation procedures.

### BACKGROUND OF THE INVENTION

Collagen has been widely used in numerous medical and cosmetic applications, including soft tissue augmentation procedures. Collagen used in these applications is endogenous collagen typically extracted from natural sources, such as animal, particularly bovine and porcine, or human tissues. Bovine collagen, in particular, has gained widespread use as an implant material for soft tissue augmentation.

Persistence is a critical feature of material to be implanted and used in various tissue augmentation procedures. The collagen that is the main bulking and structural material in most tissue augmentation products is susceptible upon implantation to degradation by host collagenases, and thus tends to degrade over a fairly short period of time. Lack of persistence due to degradation or resorption of the material can impair or compromise desired results, and retreatment can be required. Therefore, patients in need of or desiring continuous treatment or effects of longer duration must receive additional injections as early as the second or third month post-injection. (See, e.g., L. Baumann and E. Weisberg, “Soft Tissue Augmentation,” Chapter 19, in Cosmetic Dermatology Principles and Practice, pp. 155-172, 2002, McGraw Hill, NY, N.Y., which discusses the impermanence of commercially available dermal filler products, and lists the duration of, e.g., ZYPLAST and ZYDERM bovine collagen dermal fillers (Inamed Corp., Santa Barbara, Calif.) at 2-4 months.) In the case of voluntary cosmetic procedures, the inconvenience, cost, and discomfort associated with a particular procedure can discourage prospective patients from obtaining retreatment, or from choosing to undergo a procedure in the first place, knowing the desired effects will be short-lived.

Various modifications directed to overcoming the lack of persistence/short duration of tissue-derived collagen bulking agents include both efforts to alter the physical properties of extracted collagen, e.g., by chemical crosslinking of the collagen source material (use of chemical agents to impose additional crosslinks on extracted collagen) (see, e.g., Narins et al. (2003) Dermatol. Surg. 29:588-595), and by increasing the concentration of collagen in the augmentation or bulking product (see, e.g., U.S. Pat. No. 5,428,024). However, these formulations result in collagen with compromised/reduced manipulability, extrudability, and intrudability. Therefore, there is a need in the art for a collagen material having increased persistence for use in various medical and cosmetic applications. There is a further need for a collagen material that has increased persistence, with no concomitant reduction in or compromise of manipulability, extrudability, and intrudability.

The present invention addresses these needs by providing implantable collagen compositions having improved persistence, e.g., collagen compositions with persistence greater than that of commercially available collagen-based tissue augmentation products. Implantable collagen materials with minimal or no variability are also provided. The present invention further provides collagen compositions having improved manipulability, extrudability, and intrudability.

### SUMMARY OF THE INVENTION

The present invention relates to various implantable compositions comprising collagen and offering enhanced persistence, improved manipulability and ease of handling, and uniformity and reproducibility. These compositions are suitable for use in various medical and cosmetic procedures, and, in particular, in tissue augmentation procedures. Methods for producing and for using these implantable compositions are provided, as are kits comprising them.

For purposes of the present invention, an implantable composition is a composition that can be administered to a tissue to support, augment, strengthen, or enhance the tissue. In particular, it is contemplated that an implantable composition is one suitable for in vivo administration and for use as an implant or implantable device. The implantable compositions of the present invention include injectable compositions, e.g., compositions that can be administered by injection. It is further contemplated that the implantable compositions of the present invention are suitable for subcutaneous, intradermal, or subdermal administration, and are suitable for use in soft tissue augmentation and hard tissue augmentation.

In one aspect, the present invention provides an implantable composition comprising collagen, wherein the collagen consists of type III collagen. In a particular aspect, the type III collagen is human type III collagen. In another aspect, the type III collagen is synthetic type III collagen. In yet another aspect, the type III collagen is substantially free of any endogenous crosslinks. In a further aspect, the type III collagen is substantially free of any intramolecular and intermolecular crosslinks. In certain aspects, the type III collagen comprises the amino acid sequence of SEQ ID NO:1 or collagenous, e.g., triple helical, fragments thereof. In other aspects, the type III collagen comprises amino acid residue 149 to amino acid residue 1221 of the amino acid sequence of SEQ ID NO:1 or collagenous, e.g., triple helical, fragments thereof. In yet other aspects, the type III collagen comprises amino acid residue 168 to amino acid residue 1196 of the amino acid sequence of SEQ ID NO: 1 or collagenous, e.g., triple helical, fragments thereof.

In various aspects, the implantable composition comprising collagen, wherein the collagen consists of type III collagen, is an injectable composition, e.g., is suitable for delivery by injection. It is contemplated in some aspects that the delivery is subcutaneous, intradermal, or subdermal. In particular aspects, the implantable composition is selected from the group consisting of a dermal filler, a bone-void filler, and a dental implant. The invention further encompasses aspects in which the implantable composition is suitable for use in tissue augmentation, and particular aspects in which the tissue augmentation is soft tissue augmentation and in which the tissue augmentation is hard tissue augmentation.

The invention specifically provides compositions comprising collagen, wherein the collagen comprises at least a certain percentage of type III collagen. In one embodiment, the invention encompasses an implantable composition comprising collagen, wherein the collagen comprises at least 60% type III collagen. Specific embodiments in which the collagen comprises at least 70% type III collagen and at least 80% type III collagen, respectively, are contemplated, as are preferred embodiments in which the collagen comprises at least 90%, at least 95%, and at least 98% type Im collagen. In various embodiments, implantable compositions comprising certain percentages of type III collagen are provided, wherein the type III collagen is human type III collagen, is synthetic type III collagen, is substantially free of any intermolecular and intramolecular crosslinks, or is substantially free of any endogenous crosslinks. In some embodiments, the type III collagen comprises the amino acid sequence of SEQ ID NO:1 or collagenous, e.g., triple helical, fragments thereof. In other embodiments, the type III collagen comprises amino acid residue 149 to amino acid residue 1221 of the amino acid sequence of SEQ ID NO:1 or collagenous, e.g., triple helical, fragments thereof. In yet other embodiments, the type III collagen comprises amino acid residue 168 to amino acid residue 1196 of the amino acid sequence of SEQ ID NO:1 or collagenous, e.g., triple helical, fragments thereof. In various embodiments, the implantable composition is an injectable composition, e.g., is suitable for delivery by injection. In further aspects, the delivery is subcutaneous, intradermal, or subdermal.

In particular embodiments, the implantable composition comprising at least a certain percentage of type III collagen is an implantable composition selected from the group consisting of a dermal filler, a bone-void filler, and a dental implant. In preferred embodiments, the implantable composition is suitable for use in tissue augmentation. Embodiments in which the tissue augmentation is soft tissue augmentation and in which the tissue augmentation is hard tissue augmentation are specifically contemplated.

An implantable composition comprising collagen, wherein the collagen is substantially free of intermolecular and intramolecular crosslinks, is contemplated herein. In one aspect, the collagen consists of type III collagen. In other aspects, the collagen comprises at least a certain percentage of type III collagen, for example, at least 60%, at least 70%, at least 80%, at least 90%, at least 95%, or at least 98% type im collagen, respectively. In one aspect, the collagen is fibrillar collagen, and, in particular aspects, is selected from the group consisting of type I collagen, type II collagen, and type III collagen. In a preferred aspect, the collagen is human collagen; in a most preferred aspect, human type III collagen. It is contemplated that, in various aspects, the collagen is synthetic collagen. In a particular aspects, the collagen comprises the amino acid sequence of SEQ ID NO:1 or collagenous, e.g., triple helical, fragments thereof. In other aspects, the type III collagen comprises amino acid residue 149 to amino acid residue 1221 of the amino acid sequence of SEQ ID NO:1 or collagenous, e.g., triple helical, fragments thereof. In yet other aspects, the type III collagen comprises amino acid residue 168 to amino acid residue 1196 of the amino acid sequence of SEQ ID NO:1 or collagenous, e.g., triple helical, fragments thereof.

The invention further provides an implantable composition comprising collagen substantially free of intramolecular and intermolecular crosslinks, wherein the implantable composition is selected from the group consisting of a dermal filler, a bone-void filler, and a dental implant. In specific embodiments, the implantable composition is suitable for use in tissue augmentation. Embodiments in which the tissue augmentation is soft tissue augmentation and in which the tissue augmentation is hard tissue augmentation are specifically contemplated.

Implantable compositions comprising collagen, wherein the collagen is substantially free of endogenous crosslinks, are contemplated by the present invention. Collagens substantially free of endogenous crosslinks may include but are not limited to collagens that, while free of endogenous crosslinks, have been exposed subsequent to endogenous production to crosslinking agents (chemical crosslinking agents, e.g., gluteraldehyde, etc.) or to crosslinking conditions (e.g., heat, radiation, etc.), such that the collagen does contain non-endogenously imposed crosslinks.

In one embodiment, the collagen substantially free of endogenous crosslinks is fibrillar collagen. In various embodiments, the collagen is selected from the group consisting of type I collagen, type II collagen, and type III collagen. In other embodiments, the collagen substantially free of endogenous crosslinks comprises at least a certain percentage of type III collagen, for example, at least 60%, at least 70%, at least 80%, at least 90%, at least 95%, or at least 98% type III collagen, respectively. In a particular embodiment, the collagen is synthetic collagen. In one embodiment, the collagen is human collagen. In another embodiment, the collagen comprises the amino acid sequence of SEQ ID NO:1 or collagenous, e.g., triple helical, fragments thereof. In other embodiments, the type III collagen comprises amino acid residue 149 to amino acid residue 1221 of the amino acid sequence of SEQ ID NO:1 or collagenous, e.g., triple helical, fragments thereof. In yet other embodiments, the type III collagen comprises amino acid residue 168 to amino acid residue 1196 of the amino acid sequence of SEQ ID NO:1 or collagenous, e.g., triple helical, fragments thereof.

The invention further provides an implantable composition comprising collagen substantially free of intramolecular and intermolecular crosslinks, wherein the implantable composition is selected from the group consisting of a dermal filler, a bone-void filler, and a dental implant. In specific embodiments, the implantable composition is suitable for use in tissue augmentation. Embodiments in which the tissue augmentation is soft tissue augmentation and in which the tissue augmentation is hard tissue augmentation are specifically contemplated.

In one aspect, the present invention provides a method for augmenting soft tissue in a subject, the method comprising administering to the subject at an augmentation site a composition comprising collagen, wherein the collagen consists of type III collagen, thereby augmenting the soft tissue. In one aspect, the subject is a mammal. In a preferred aspect, the subject is a human. In another aspect, the type III collagen is of the same species as the subject. In a particular aspect, the type III collagen is human type III collagen and the subject is a human. In another aspect, the type III collagen is synthetic type III collagen. In a preferred aspect, the type III collagen is synthetic human type III collagen. In certain aspects, the type III collagen comprises the sequence of SEQ ID NO:1 or collageneous, e.g., triple helical, fragements thereof. In other aspects, the type III collagen comprises amino acid residue 149 to amino acid residue 1221 of the amino acid sequence of SEQ ID NO:1 or collagenous, e.g., triple helical, fragments thereof. In yet other aspects, the type III collagen comprises amino acid residue 168 to amino acid residue 1196 of the amino acid sequence of SEQ ID NO:1 or collagenous, e.g., triple helical, fragments thereof.

In various aspects, the administering to the subject at an augmentation site is by implantation or injection, e.g., using a syringe and needle, etc. In certain aspects, the administering to the subject at an augmentation site is subcutaneous administration, intradermal administration, or subdermal administration.

In one embodiment, the present invention provides a method for augmenting soft tissue in a subject, the method comprising administering to the subject at an augmentation site a composition comprising collagen, wherein the collagen comprises at least 60% type III collagen, thereby augmenting the soft tissue. In one embodiment, the subject is a mammal. In a preferred embodiment, the subject is a human. Specific embodiments in which the collagen comprises at least 70% type III collagen and at least 80% type III collagen, respectively, are contemplated, as are preferred embodiments in which the collagen comprises at least 90%, at least 95%, and at least 98% type III collagen. In another embodiment, the type III collagen is of the same species as the subject. In a particular embodiment, the type III collagen is human type III collagen and the subject is a human. In another embodiment, the type III collagen is synthetic type III collagen. In a preferred embodiment, the type III collagen is synthetic human type III collagen. In certain embodiments, the type III collagen comprises the sequence of SEQ ID NO:1 or collageneous, e.g., triple helical, fragements thereof. In other embodiments, the type III collagen comprises amino acid residue 149 to amino acid residue 1221 of the amino acid sequence of SEQ ID NO:1 or collagenous, e.g., triple helical, fragments thereof. In yet other embodiments, the type III collagen comprises amino acid residue 168 to amino acid residue 1196 of the amino acid sequence of SEQ ID NO:1 or collagenous, e.g., triple helical, fragments thereof.

In various embodiments, the administering to the subject at an augmentation site is by implantation or injection, e.g., using a syringe and needle, etc. In certain aspects, the administering to the subject at an augmentation site is subcutaneous administration, intradermal administration, or subdermal administration.

In one aspect, the present invention provides a method for augmenting soft tissue in a subject, the method comprising administering to the subject at an augmentation site a composition comprising collagen, wherein the collagen is substantially free of intermolecular and intramolecular crosslinks, thereby augmenting the soft tissue. In one aspect, the subject is a mammal. In a preferred aspect, the subject is a human. In another aspect, the collagen is fibrillar collagen, and, in particular aspects, is selected from the group consisting of type I collagen, type II collagen, and type III collagen. In other aspects, the collagen comprises at least a certain percentage of type III collagen, for example, at least 60%, at least 70%, at least 80%, at least 90%, at least 95%, or at least 98% type III collagen, respectively. In a preferred aspect, the collagen is human collagen; in a most preferred aspect, human type III collagen. It is contemplated that, in various aspects, the collagen is synthetic collagen. In a particular aspect, the collagen comprises the sequence of SEQ ID NO:1 or collagenous, e.g., triple helical, fragments thereof. In other aspects, the type III collagen comprises amino acid residue 149 to amino acid residue 1221 of the amino acid sequence of SEQ ID NO:1 or collagenous, e.g., triple helical, fragments thereof. In yet other aspects, the type III collagen comprises amino acid residue 168 to amino acid residue 1196 of the amino acid sequence of SEQ ID NO:1 or collagenous, e.g., triple helical, fragments thereof.

In various aspects, the administering to the subject at an augmentation site is by implantation or injection, e.g., using a syringe and needle, etc. In certain aspects, the administering to the subject at an augmentation site is subcutaneous administration, intradermal administration, or subdermal administration.

In one embodiment, the present invention provides a method for augmenting soft tissue in a subject, the method comprising administering to the subject at an augmentation site a composition comprising collagen, wherein the collagen is substantially free of endogenous crosslinks, thereby augmenting the soft tissue. In one embodiment, the subject is a mammal. In a preferred embodiment, the subject is a human. Collagens substantially free of endogenous crosslinks may include but are not limited to collagens that, while free of endogenous crosslinks, have been exposed subsequent to endogenous production to crosslinking agents (chemical crosslinking agents, e.g., gluteraldehyde, etc.) or to crosslinking conditions (e.g., heat, radiation, etc.), such that the collagen does contain non-endogenously imposed crosslinks. In one embodiment, the collagen is fibrillar collagen, and, in particular embodiments, is selected from the group consisting of type I collagen, type II collagen, and type III collagen. In other embodiments, the collagen comprises at least a certain percentage of type III collagen, for example, at least 60%, at least 70%, at least 80%, at least 90%, at least 95%, or at least 98% type III collagen, respectively. In a preferred embodiment, the collagen is human collagen; in a most preferred embodiment, human type III collagen. It is contemplated that, in various embodiments, the collagen is synthetic collagen. In a particular embodiment, the collagen comprises the sequence of SEQ ID NO:1 or collagenous, e.g., triple helical, fragments thereof. In other embodiments, the type III collagen comprises amino acid residue 149 to amino acid residue 1221 of the amino acid sequence of SEQ ID NO:1 or collagenous, e.g., triple helical, fragments thereof. In yet other embodiments, the type III collagen comprises amino acid residue 168 to amino acid residue 1196 of the amino acid sequence of SEQ ID NO:1 or collagenous, e.g., triple helical, fragments thereof.

In various aspects, the administering to the subject at an augmentation site is by implantation or injection, e.g., using a syringe and needle, etc. In certain aspects, the administering to the subject at an augmentation site is subcutaneous administration, intradermal administration, or subdermal administration.

In one embodiment, the present invention provides a method for preparing an implantable collagen composition, the method comprising providing a collagen, wherein the collagen consists of type III collagen, and preparing an aqueous solution of the type III collagen, thereby preparing an implantable collagen composition. In one embodiment, the type III collagen is human type III collagen. In another embodiment, the type III collagen is synthetic type III collagen. In a preferred embodiment, the type III collagen is synthetic human type III collagen. In yet another embodiment, the type III collagen is substantially free of any intermolecular and intramolecular crosslinks. In a further embodiment, the type III collagen is substantially free of any endogenous crosslinks. In certain embodiments, the type III collagen comprises the sequence of SEQ ID NO:1 or collagenous, e.g., triple helical, fragments thereof. In other embodiments, the type III collagen comprises amino acid residue 149 to amino acid residue 1221 of the amino acid sequence of SEQ ID NO:1 or collagenous, e.g., triple helical, fragments thereof. In yet other embodiments, the type III collagen comprises amino acid residue 168 to amino acid residue 1196 of the amino acid sequence of SEQ ID NO:1 or collagenous, e.g., triple helical, fragments thereof.

In one aspect, the present invention provides a method for preparing an implantable collagen composition, the method comprising providing a collagen, wherein the collagen comprises at least 60% type III collagen, and preparing an aqueous solution of the collagen, thereby preparing an implantable collagen composition. Specific aspects in which the collagen comprises at least 70% type III collagen and at least 80% type III collagen, respectively, are contemplated, as are preferred aspects in which the collagen comprises at least 90%, at least 95%, and at least 98% type III collagen. In certain aspects, the collagen is human type III collagen. In other aspects, the collagen is synthetic type III collagen. In preferred aspects, the collagen is synthetic human type III collagen. In yet another aspect, the collagen is substantially free of any intermolecular and intramolecular crosslinks. In a further aspect, the collagen is substantially free of any endogenous crosslinks. In certain aspects, the type III collagen comprises the sequence of SEQ ID NO:1 or collagenous, e.g., triple helical, fragments thereof. In other aspects, the type III collagen comprises amino acid residue 149 to amino acid residue 1221 of the amino acid sequence of SEQ ID NO:1 or collagenous, e.g., triple helical, fragments thereof. In yet other aspects, the type III collagen comprises amino acid residue 168 to amino acid residue 1196 of the amino acid sequence of SEQ ID NO:1 or collagenous, e.g., triple helical, fragments thereof.

In one embodiment, the present invention provides a method for preparing an implantable collagen composition, the method comprising providing a collagen substantially free of intermolecular and intramolecular crosslinks, and preparing an aqueous solution of the collagen substantially free of intermolecular and intramolecular crosslinks, thereby preparing an implantable collagen composition. In one embodiment, the collagen is fibrillar collagen, and, in particular embodiments, is selected from the group consisting of type I collagen, type II collagen, and type III collagen. In certain embodiments, the collagen is human collagen. In other embodiments, the collagen is synthetic collagen. In preferred embodiments, the collagen is synthetic human collagen. In a preferred embodiment, the collagen is synthetic human type III collagen. In a further embodiment, the collagen is substantially free of any endogenous crosslinks. In a particular embodiment, the collagen comprises the sequence of SEQ ID NO:1 or collagenous, e.g., triple helical, fragments thereof. In other embodiments, the type III collagen comprises amino acid residue 149 to amino acid residue 1221 of the amino acid sequence of SEQ ID NO:1 or collagenous, e.g., triple helical, fragments thereof. In yet other embodiments, the type III collagen comprises amino acid residue 168 to amino acid residue 1196 of the amino acid sequence of SEQ ID NO:1 or collagenous, e.g., triple helical, fragments thereof.

The present invention also provides a method for preparing an implantable collagen composition, the method comprising providing a collagen substantially free of intermolecular and intramolecular crosslinks, and crosslinking the collagen, thereby preparing an implantable collagen composition. In one aspect, the collagen is fibrillar collagen, and, in particular aspects, is selected from the group consisting of type I collagen, type II collagen, and type III collagen. In another aspect, the collagen is human collagen. In another aspect, the collagen is synthetic collagen. In a preferred aspect, the collagen is synthetic human collagen. In a particular aspect, the collagen comprises the sequence of SEQ ID NO:1 or collagenous, e.g., triple helical, fragments thereof. In other aspects, the type III collagen comprises amino acid residue 149 to amino acid residue 1221 of the amino acid sequence of SEQ ID NO:1 or collagenous, e.g., triple helical, fragments thereof. In yet other aspects, the type III collagen comprises amino acid residue 168 to amino acid residue 1196 of the amino acid sequence of SEQ ID NO:1 or collagenous, e.g., triple helical, fragments thereof. In certain aspects, crosslinking the collagen occurs by exposing the collagen to heat or radiation under conditions suitable to crosslink the collagen. In other aspects, crosslinking the collagen occurs by exposing the collagen to a chemical crosslinking agent under conditions suitable to crosslink the collagen. In particular aspects, the chemical crosslinking agent is selected from the group consisting of aldehydes, carbodiimides, epoxides, and imidazoles.

In one embodiment, the present invention provides a method for preparing an implantable collagen composition, the method comprising providing a collagen substantially free of endogenous crosslinks, crosslinking the collagen, and preparing an aqueous solution of the crosslinked collagen, thereby preparing an implantable collagen composition. In one embodiment, crosslinking the collagen occurs by exposing the collagen to heat or radiation under conditions suitable to crosslink the collagen. In one embodiment, the collagen is fibrillar collagen, and, in particular embodiments, is selected from the group consisting of type I collagen, type II collagen, and type III collagen. In another embodiment, the collagen is human collagen. In another embodiment, the collagen is synthetic collagen. In a preferred embodiment, the collagen is synthetic human collagen. In a particular embodiment, the collagen comprises the sequence of SEQ ID NO:1 or collagenous, e.g., triple helical, fragments thereof. In other embodiments, the type III collagen comprises amino acid residue 149 to amino acid residue 1221 of the amino acid sequence of SEQ ID NO:1 or collagenous, e.g., triple helical, fragments thereof. In yet other embodiments, the type III collagen comprises amino acid residue 168 to amino acid residue 1196 of the amino acid sequence of SEQ ID NO:1 or collagenous, e.g., triple helical, fragments thereof. In another embodiment, crosslinking the collagen occurs by exposing the collagen to a chemical crosslinking agent under conditions suitable to crosslink the collagen. In certain embodiments, the chemical crosslinking agent is selected from the group consisting of aldehydes, carbodiimides, epoxides, and imidazoles.

In one aspect, the present invention provides a kit useful for augmenting soft tissue comprising an implantable collagen composition, the kit comprising type III collagen, a syringe, and a needle. In one aspect, the type III collagen is human type III collagen. In another aspect, the type III collagen is synthetic type III collagen. In a preferred aspect, the type III collagen is synthetic human type III collagen. In yet another aspect, the type III collagen is substantially free of any intermolecular and intramolecular crosslinks. In a further aspect, the type III collagen is substantially free of any endogenous crosslinks. In certain aspects, the type III collagen comprises the sequence of SEQ ID NO:1 or collagenous, e.g., triple helical, fragments thereof. In other aspects, the type III collagen comprises amino acid residue 149 to amino acid residue 1221 of the amino acid sequence of SEQ ID NO:1 or collagenous, e.g., triple helical, fragments thereof. In yet other aspects, the type III collagen comprises amino acid residue 168 to amino acid residue 1196 of the amino acid sequence of SEQ ID NO:1 or collagenous, e.g., triple helical, fragments thereof.

In one embodiment, the present invention provides a kit useful for augmenting soft tissue comprising an implantable collagen composition, the kit comprising a collagen, wherein the collagen comprises at least 60% type III collagen, a syringe, and a needle. Specific embodiments in which the collagen comprises at least 70% type III collagen and at least 80% type III collagen, respectively, are contemplated, as are preferred embodiments in which the collagen comprises at least 90%, at least 95%, and at least 98% type III collagen. In certain embodiments, the collagen is human type III collagen. In other embodiments, the collagen is synthetic type III collagen. In preferred embodiments, the collagen is synthetic human type III collagen. In yet another embodiment, the collagen is substantially free of any intermolecular and intramolecular crosslinks. In a further embodiment, the collagen is substantially free of any endogenous crosslinks. In certain embodiments, the type III collagen comprises the sequence of SEQ ID NO:1 or collagenous, e.g., triple helical, fragments thereof. In other embodiments, the type III collagen comprises amino acid residue 149 to amino acid residue 1221 of the amino acid sequence of SEQ ID NO:1 or collagenous, e.g., triple helical, fragments thereof. In yet other embodiments, the type III collagen comprises amino acid residue 168 to amino acid residue 1196 of the amino acid sequence of SEQ ID NO:1 or collagenous, e.g., triple helical, fragments thereof.

In one aspect, the present invention provides a kit useful for augmenting soft tissue comprising an implantable collagen composition, the kit comprising a collagen, wherein the collagen is substantially free of intermolecular and intramolecular crosslinks, a syringe, and a needle. In one aspect, the collagen is fibrillar collagen, and, in particular aspects, is selected from the group consisting of type I collagen, type II collagen, and type III collagen. In certain aspects, the collagen is human collagen. In other aspects, the collagen is synthetic collagen. In preferred aspects, the collagen is synthetic human collagen. In a preferred aspects, the collagen is synthetic human type III collagen. In a further aspect, the collagen is substantially free of any endogenous crosslinks. In a particular aspect, the collagen comprises the sequence of SEQ ID NO:1 or collagenous, e.g., triple helical, fragments thereof. In other embodiments, the type III collagen comprises amino acid residue 149 to amino acid residue 1221 of the amino acid sequence of SEQ ID NO:1 or collagenous, e.g., triple helical, fragments thereof. In yet other embodiments, the type III collagen comprises amino acid residue 168 to amino acid residue 1196 of the amino acid sequence of SEQ ID NO:1 or collagenous, e.g., triple helical, fragments thereof.

### BRIEF DESCRIPTION OF THE DRAWINGS

FIGS. 1A and 1B show SDS-PAGE analysis of synthetic human type I collagen and synthetic human type III collagen following treatment with matrix metalloproteinase (MMP)-1 (FIG. 1A) and MMP-8 (FIG. 1B).

### DESCRIPTION OF THE INVENTION

The present invention provides implantable collagen compositions useful for tissue augmentation applications. The implantable collagen compositions of the present invention have increased persistence suitable for use in various medical and cosmetic applications. Implantable collagen compositions with minimal or no variability and with improved manipulability, extrudability, and intrudability are also provided.

The present invention relates in part to the discovery that type III collagen displays greater persistence than does type I collagen. In particular, the present inventors discovered that implantable type III collagen compositions have persistence greater than that of implantable type I collagen compositions, e.g., type III collagen will persist longer and degrade at a slower rate than type I collagen. Therefore, in one embodiment, the present invention provides an implantable composition comprising collagen, wherein the collagen consists of type III collagen. In other embodiments, the present invention provides implantable compositions that comprise collagen, wherein the collagen comprises a specific and predetermined amount of type III collagen, sufficient to give increased persistence to the final product.

The determination of whether the amount of type III collagen in a particular composition is sufficient to give that composition increased persistence can be measured, e.g., by evaluating the longevity of a composition containing a specific percentage of type III collagen, such as by visual or palpable assessment, for example, using Global Aesthetic Improvement Scale (GAIS) ratings, or by assessing in vitro resistance to metalloprotease degradation (see, e.g., Example 2), etc. GAIS is based on a physician's assessment of the overall improvement, e.g., cosmetic improvement, in a treated area, e.g., nasolabial fold, by comparing the patient's appearance after treatment to that before treatment. GAIS ratings include: very much improved (optimal cosmetic result for the implant in the patient); much improved (marked improvement in appearance from the initial condition, but not completely optimal for this patient); improved (obvious improvement in appearance from initial condition); no change (the appearance is essentially the same as the original condition); and worse (the appearance is worse than the original condition). In various embodiments, the amount of type III collagen in the implantable collagen composition is greater than 60%, greater than 70%, and greater than 80% type III collagen. In preferred embodiments, the amount of type III collagen in the implantable collagen composition is greater than 90%; more preferably, greater than 95%; and, most preferably, greater than 98% type im collagen.

No current products provide a collagen component that consists of type III collagen or that consists of a predetermined and reproducible amount of type III collagen sufficient to provide increased persistence. Collagen used as a tissue bulking or augmenting material in various procedures is endogenous collagen, either extracted from animal, typically bovine tissue, e.g., by dispersion (mechanically shearing the tissue), dissolution (acid cleavage), enzymatic digestion, etc., or endogenously produced, e.g., by cultured fibroblasts. Endogenous collagen is a variable mixture of collagen types, typically containing 95% type I collagen, e.g., the predominant collagen type in bones and tissues, approximately 5% type III collagen, as well as trace amounts of other collagen types.

Endogenous human collagen can be similarly obtained from skin derived from the patient or from cadavers. (See, e.g., U.S. Pat. No. 4,969,912 and U.S. Pat. No. 5,116,389.) Like tissue-extracted bovine collagen, extracted human collagen is a mixture of collagen types; for example, human skin collagen contains about 95% type I collagen, 5% type III collagen, and trace amounts of other collagen types. The amounts of collagen thus obtained are limited, and cost and consumer distaste are prohibitive. Human collagen has also been obtained from human fibroblast cells grown in a controlled laboratory environment. (See, e.g., COSMODERM and COSMOPLAST formulations, Inamed Corp., Santa Barbara Calif.) These cultured fibroblasts behave as if they were in the human body, and produce natural collagen, e.g., the collagen normally found in human skin, e.g., a mixture of approximately 95% type I collagen, 5% type III collagen, and trace amounts of other collagen types. The collagen thus obtained is endogenous material, and production varies based on growth conditions and occurs in relatively limited amounts.

The present invention further relates to the unexpected finding that collagen compositions containing collagen substantially free of intermolecular and intramolecular crosslinks are useful as implantable materials.

All collagen currently used in tissue augmentation products is endogenous collagen, e.g., extracted from animal, e.g., bovine or human, tissues or endogenously produced, for example, by cultured fibroblasts. Endogenous collagens are subject to extensive post-translational modifications. (Kivirikko (1998) Matrix Biol 16:355-356.) In particular, the assembly and processing of collagens, such as the fibrillar collagens (e.g., type I, type II, and type III collagen), is accompanied by the formation of intramolecular and intermolecular covalent crosslinks, i.e., crosslinks that form intramolecularly between the individual collagen α-chains forming a triple helical collagen molecule, or intermolecularly between triple helical collagen molecules, enhancing fibrillar stability. Natural crosslinks form between hydroxylysine and lysine residues or between hydroxylysine, lysine, and histidine residues of the collagen molecules.

Crosslinks confer tensile and mechanical strength and contribute to tissue integrity and are associated with resistance of the collagen to degradation by host collagenases, proteases, etc. Therefore, it has been believed in the art that a degree of natural or endogenous crosslinking was required in order for collagen to be useful as an implant material having an appropriate amount of mechanical and structural function and persistence for use in various medical and cosmetic applications. Crosslinking contributes to and prolongs the structural integrity of crosslinked collagen, but also affects other physical parameters, including flowability and solubility, and limits the workable concentration of collagen in a final tissue augmentation product. Therefore, the ease of manipulation and handling of the collagen, and its suitability for injection, can be compromised by the degree and nature of crosslinking present.

The present invention shows surprisingly that collagen substantially free of crosslinks, e.g., substantially free of intermolecular and intramolecular crosslinks, demonstrated persistence and resistance to degradation and thus suitability for use in various tissue augmentation applications. (See Example 2 and Example 3.) Therefore, in one embodiment, the present invention provides an implantable composition containing collagen, wherein the collagen is substantially free of intramolecular and intermolecular crosslinks. This offers unique advantages over currently available products, as collagen substantially free of intermolecular or intramolecular crosslinks is not affected by the loss of or reduction in manipulability, extrudability, and intrudability characteristic of crosslinked material. In one embodiment, the collagen is a fibrillar collagen. In further embodiments, the collagen is selected from the group consisting of type I collagen and type III collagen. In a preferred embodiment, the collagen is type III collagen. In various embodiments, the collagen comprises various percentages of type III collagen. In specific embodiments, the amount of type III collagen in the implantable composition is greater than 60% type III collagen, greater than 70% type III collagen, and greater than 80% type III collagen. In preferred embodiments, the amount of type III collagen in the implantable composition is greater than 90% type III collagen; more preferably, greater than 95% type III collagen; and, most preferably, greater than 98% type III collagen.

While the present invention provides implantable compositions comprising collagen substantially free of intramolecular and intermolecular crosslinks, implantable compositions comprising collagens free of endogenous crosslinks, but containing non-endogenously imposed crosslinks, are specifically contemplated herein.

As crosslinks have been traditionally associated with increased stability and persistence of implanted collagen material, various current collagen-containing products involve the use of chemically or other exogenously imposed crosslinks to further enhance the persistence of the collagen material. (See McPherson et al. (1986) J Biomed Mater Res 20:93-107.) For example, clinical studies using a commercially available injectable bovine collagen (i.e., ZYDERM) demonstrated that, while efficacious at correcting facial wrinkles, the clinical correction was temporary. (See Burke et al. (1985) Ann Plast Surg 14:515-522; Rapaport et al. (1984) J Am Acad Dermatol 11(2 Pt 1):25-252.) This prompted the development of the more stable injectable material ZYPLAST, a gluteraldehyde crosslinked injectable bovine collagen, which is a more persistent material than ZYDERM and thus provides a more durable aesthetic effect. Therefore, as taught in the art, implantable biomaterials containing exogenously imposed crosslinks are associated with a more stable and durable material and with increased persistence of the material.

These additional or non-endogenous crosslinks are introduced to the collagen material, e.g., by exposing the collagen to chemical crosslinking agents, radiation, etc. As noted, supra, an increased degree of crosslinking may enhance persistence but can compromise other aspects of performance of the collagen material, including ease of handling, such that in some cases the collagen requires additional processing and treatment to render it suitable for injection. (See, e.g., U.S. Pat. No. 4,582,640.)

It is an advantage of the present material that, in one aspect, the present invention provides a starting collagen material substantially free of any intramolecular or intermolecular crosslinks. This collagen can be exposed to crosslinking agents or conditions suitable to induce crosslinking (e.g., gluteraldehyde, radiation, etc.), resulting in collagen possessing a degree of crosslinking that is sufficient to enhance persistence, but that does not compromise the ease of handling of the material. In particular, a collagen that is substantially free of any endogenous intermolecular or intramolecular crosslinks, and that is subsequently crosslinked, e.g., by use of chemical agent or exposure to crosslinking conditions such as radiation, can be crosslinked to a lesser extent than an endogenously crosslinked collagen subsequently crosslinked under the same conditions.

In one aspect, the invention provides an optimal starting material for manufacture of an implantable collagen composition, wherein the starting material is or comprises a collagen substantially free of endogenous crosslinks. While the invention specifically contemplates, in various aspects, that the manufacture of an implantable composition suitable for use in various cosmetic and medical procedures, including tissue augmentation procedures, will involve crosslinking the collagen, it is a distinct advantage of the particular compositions of the present invention that the degree and nature of the crosslinking imposed are completely within control of the manufacture. In contrast, the endogenously produced materials currently available contain endogenous crosslinks as a result of their natural production. The present invention thus, in certain embodiments, offers uniquely consistent and reproducible starting material, allowing for a predictable and controlled performance at every step in manufacturing and offering advantages over the endogenously produced materials used today.

Thus, in one aspect, the invention contemplates a method of manufacturing an implantable collagen composition, the method comprising: obtaining collagen substantially free of any intramolecular or intermolecular crosslinks, and crosslinking the collagen, thereby manufacturing an implantable collagen composition. Collagen can be crosslinked using methods known to one of skill in the art, such as by heat, radiation, or using chemical crosslinking agents available in the art, such as, for example, aldehydes, carbodiimides, epoxides, imidazoles, etc.

This is advantageous in that is allows more control and predictability with respect to the nature and degree of crosslinking of the collagen. In particular, the degree of crosslinking can be controlled, e.g., by modification of crosslinking agent concentration or radiation strength, or time of exposure to the modifying conditions. The nature, including the type, of crosslinking can be controlled, e.g., by altering conditions to favor the formation of intramolecular over intermolecular crosslinks, such as by use of particular crosslinking agents, by varying the concentration of collagen in the solution to be crosslinked, etc.

In one embodiment, the present invention provides an injectable composition comprising collagen with no endogenous crosslinks. This can include both a material that contains no intermolecular and no intramolecular crosslinks, and a material that contains no endogenous crosslinks, but does contain crosslinks introduced in a non-endogenous setting. The crosslinks introduced in a non-endogenous setting include, e.g., crosslinks resulting from exposure of the collagen to crosslinking agents, such as chemical crosslinking agents, from exposure of the collagen to radiation, etc.

In preferred embodiments, collagen used in the present compositions and methods is produced by synthetic means, most preferably by recombinant means. This can be advantageous for a number of reasons, as the use of synthetic collagen in an implantable composition of the present invention results in a characterized and fully reproducible collagen that has sufficient strength, stability, and, persistence, essentially no immunoreactivity, tissue response, or inflammatory reaction upon implantation, and that is free of pathogenic, e.g., infectious, agents, and of other contaminants, e.g., animal-derived contaminants, etc.

The use of bovine or other animal collagen (porcine, equine, etc.) as the predominant component of various tissue augmentation and implant materials carries with it the potential for adverse reaction upon implantation/injection. In particular, immune reponses including allergic reactions and non-allergic reactions, e.g., infections, etc., have been observed upon exposure to non-human collagen-containing products. (See, e.g., Baumann and Weisberg, supra, page 157.) For example, positive allergic reactions to bovine collagen are seen in 3 to 10 percent of patients undergoing skin testing. (See Barr and Stegman (1984) J Am Acad Dermatol 10: 652; Charrierre et al. (1989) J Am Acad Dermatol 21:1203.) There is also a smaller but significant subgroup of allergic patients who develop an allergy to bovine collagen even after one or two negative skin tests. These reactions can be devastating and difficult, if not impossible, to treat.

If an inflammatory response is stimulated, by exposure to an allergenic, immunogenic, or even toxic component present in the collagen source material, infiltrating inflammatory cells and fibroblasts can restructure both native and implanted matrix components, and the injected/implanted collagen can be degraded and resorbed. Therefore, in one embodiment, the implantable compositions of the present invention comprise synthetic collagen. In a further embodiment, the synthetic collagen is recombinantly manufactured, for example, produced as described, e.g., in Example 1.

In addition to minimizing the risk for adverse reactions, the use of synthetic collagen in the implantable compositions of the present invention can be advantageous in contributing to the uniformity and reproducibility of the collagen material. As discussed, supra, endogenous collagens, including those extracted from natural sources, contain a heterogenous mixture of various types of collagen. (See, e.g., Byers et al. (1974) Biochemistry 13:5243-5248; and Miller and Rhodes (1982) Methods Enzymol 82 (Pt A):33-64.) In contrast, the present invention clearly contemplates implantable compositions containing collagen of one type free of any other type of collagen, or compositions comprising specific and predetermined percentages of more than one collagen type. In a preferred embodiment, the present invention provides an implantable composition comprising collagen, wherein the collagen consists of type III collagen. In other embodiments, the invention provides implantable compositions containing collagen, wherein the collagen comprises a specific and predetermined percentage of type III collagen. Use of synthetic, including recombinant, techniques, is advantageous in producing individual collagen types in isolated and predetermined quantities and in reproducible fashion.

Furthermore, the use of synthetic techniques enables the production of collagens that can be specifically optimized for particular uses. For example, expression constructs encoding synthetic collagens suitable for use in the present invention can contain native human sequence but have specific modifications that result in expression of synthetic collagens having desired properties, e.g., deletions, alterations, substitutions, etc., that eliminate collagenase-sensitive regions, and deletions, alterations, substitutions, etc., of various regions eliciting immunogenic, e.g., antigenic and allergenic, responses, etc.

Synthetic collagens can be produced using any of the wide range of synthetic techniques well-known in the art, including, e.g., recombinant production, peptide synthesis, etc. A preferred recombinant method is described in, e.g., Vuorela et al. (1997) EMBO J 16:6702-6712; Nokelainen et al. (2001) Yeast 18:797-806; and Myllyhatju et al. (2000) Biochem Soc Trans 28:3532-3537, incorporated by reference herein in their entirety. For recombinant production, the host cells are not restricted, and can include any cell capable of recombinant protein expression. Upon expression, the synthetic collagens may be secreted into the media or local environment, and/or the host cell may retain the collagen. Retained collagen may be compartmentalized, for example in the endoplasmic reticulum, golgi, or associated vesicles in animal cells, vacuoles in plant cells, inclusion bodies in bacteria, etc. The cells expressing the synthetic collagen may be grown in culture, or may be inserted by techniques known to those skilled in the art into transgenic plants or animals and then isolated from tissues targeted for expression.

Collagen sequences from which synthetic collagens can be derived include animal collagens and, in preferred embodiments, human collagens. Nucleic acid sequences encoding collagens are available in the art. (See, e.g., Fuller and Boedtker (1981) Biochemistry 20:996-1006; Sandell et al. (1984) J Biol Chem 259:7826-34; Kohno et al. (1984) J Biol Chem 259:13668-13673; French et al. (1985) Gene 39:311-312; Metsaranta et al. (1991) J Biol Chem 266:16862-16869; Metsaranta et al. (1991) Biochim Biophys Acta 1089:241-243; Wood et al. (1987) Gene 61:225-230; Glumoff et al. (1994) Biochim Biophys Acta 1217:41-48; Shirai et al. (1998) Matrix Biology 17:85-88; Tromp et al. (1988) Biochem J 253:919-912; Kuivaniemi et al. (1988) Biochem J 252:633-640; Ala-Kokko et al. (1989) Biochem J 260:509-516; International Publication No. WO 01/34647.) Further, the collagens used in the present formulations may comprise collagen fragments of defined length produced by direct recombinant expression of polynucleotides encoding the collagen fragment, or collagen fragments obtained by processing full-length collagen protein, e.g., by enzymatic digestion, thermal denaturation, chemical degradation, etc. Collagen fragments can include any polypeptide comprising at least a portion of the triple helical domain of a collagen or collagen-like molecule. In a preferred embodiment, the present formulations comprise synthetic collagen or synthetic collagen fragments derived from type III collagen; in a most preferred embodiment, from human type III collagen. Each synthetic collagen produced by the methods described above can be obtained as a single type of collagen, free of any other type of collagen.

In one embodiment, the present invention provides an implantable composition containing collagen, wherein the collagen is type III collagen. In another embodiment, the type III collagen is human type III collagen. In one embodiment, the present invention provides a collagen comprising the amino acid sequence of SEQ ID NO:1 or a collagenous fragment thereof. In one embodiment, the collagen comprises a polypeptide having an amino acid sequence containing the N-telopeptide domain, the α-helical domain, and the C-telopeptide domain of type III collagen. The N-telopeptide of type III collagen is from amino acid residue 149 to amino acid residue 167 of SEQ ID NO:1. The α-helical domain of type III collagen is from amino acid residue 168 to amino acid residue 1196 of SEQ ID NO:1. The C-telopeptide of type III collagen is from amino acid residue 1197 to amino acid residue 1221 of SEQ ID NO:1. In one embodiment, the collagen useful in the present methods and compositions comprises a polypeptide having an amino acid sequence of amino acid residue 149 to amino acid residue 1221 of SEQ ID NO:1. In another embodiment, the collagen has an amino acid sequence of from about amino acid residue 149 to amino acid residue 1221 of SEQ ID NO:1. In addition, an implantable composition containing collagen, wherein the collagen is a synthetic human type III collagen which contains an isoleucine to proline substitution at amino acid residue 1205 of the human type III collagen sequence of SEQ ID NO:1, is specifically contemplated herein.

In another embodiment, the collagen comprises a polypeptide having an amino acid sequence containing the α-helical domain of type III collagen. In one embodiment, the collagen useful in the compositions and methods of the present invention comprises a polypeptide having an amino acid sequence of from amino acid residue 168 to amino acid residue 1196 of SEQ ID NO:1. It is further contemplated that in certain embodiments of the present invention, the collagen for use in the present methods and compositions comprises a polypeptide having an amino acid sequence of about amino acid residue 168 to amino acid residue 1196 of SEQ ID NO:1.

In one embodiment, the present invention provides an implantable composition containing collagen, wherein the collagen is type I collagen. In one embodiment, the type I collagen is human type I collagen. In another embodiment, the present invention provides a collagen comprising the amino acid sequence of SEQ ID NO:2 or a collagenous fragment thereof. In another embodiment, the present invention provides a collagen comprising the amino acid sequence of SEQ ID NO:3 or a collagenous fragment thereof. In another embodiment, the collagen comprises a polypeptide having an amino acid sequence containing the N-telopeptide domain, the α-helical domain, and the C-telopeptide domain of type I collagen. The N-telopeptide of α1(I) collagen is from amino acid residue 162 to amino acid residue 178 of SEQ ID NO:2. The α-helical domain of α1(I) collagen is from amino acid residue 179 to amino acid residue 1192 of SEQ ID NO:2. The C-telopeptide of α1(I) collagen is from amino acid residue 1193 to amino acid residue 1218 of SEQ ID NO:2. In one embodiment, the collagen useful in the present methods and compositions comprises a polypeptide having an amino acid sequence of amino acid residue 162 to amino acid residue 1218 of SEQ ID NO:2. The N-telopeptide of α2(I) collagen is from amino acid residue 180 to amino acid residue 190 of SEQ ID NO:3. The α-helical domain of α2(I) collagen is from amino acid residue 191 to amino acid residue 1102 of SEQ ID NO:3. The C-telopeptide of α2(I) collagen is from amino acid residue 1103 to amino acid residue 1119 of SEQ ID NO:3. In one embodiment, the collagen useful in the present methods and compositions comprises a polypeptide having an amino acid sequence of amino acid residue 180 to amino acid residue 1119 of SEQ ID NO:3. In other embodiments, the collagen has an amino acid sequence selected from the group consisting of about amino acid residue 162 to amino acid residue 1218 of SEQ ID NO:2 and about amino acid residue 180 to amino acid residue 1119 of SEQ ID NO:3.

In another embodiment, the collagen comprises a polypeptide having an amino acid sequence containing the α-helical domain of type I collagen. In one embodiment, the collagen useful in the compositions and methods of the present invention comprises a polypeptide having an amino acid sequence of from amino acid residue 179 to amino acid residue 1192 of SEQ ID NO:2. In one embodiment, the collagen useful in the compositions and methods of the present invention comprises a polypeptide having an amino acid sequence of from amino acid residue 191 to amino acid residue 1102 of SEQ ID NO:3. It is further contemplated that in certain embodiments of the present invention, the collagen for use in the present methods and compositions comprises a polypeptide having an amino acid sequence selected from the group consisting of about amino acid residue 179 to amino acid residue 1192 of SEQ ID NO:2 and about amino acid residue 191 to amino acid residue 1102 of SEQ ID NO:3.

The implantable compositions provided herein can be used in any method known or contemplated by those skilled in the art. In particular, the present compositions can be used in any of the numerous medical and cosmetic applications, including tissue augmentation procedures, in which collagen is currently used and in which compositions containing collagen and having greater persistence, improved handling, and/or less variability may be desired. The present compositions are suitable for use in tissue augmentation procedures. Use of the present compositions in cosmetic as well as in medical procedures is specifically contemplated.

In one aspect, the present invention provides implantable compositions containing collagen and suitable for use in soft tissue augmentation procedures. The present compositions can be implanted or injected into various regions of the skin or dermis, depending on the particular application or cosmetic procedure, including dermal, intradermal, and subcutaneous injection or implantation. The materials of the present invention can also be injected or implanted superficially, such as, for example, within the papillary layer of the dermis, or can be injected or implanted within the reticular layer of the dermis.

In addition to soft tissue augmentation, use of the implantable compositions containing collagen for hard tissue augmentation is contemplated in the present invention. The present collagen compositions are useful in various hard tissue augmentation applications, including, for example, as a bone-void filler, dental implant, etc.

Cosmetic uses of the compositions of the present invention include treatment of fine lines, such as fine superficial facial lines, wrinkles, and scars, as well as treatment of pronounced lines, wrinkles, and scars. In some aspects, the compositions of the present invention are used for other cosmetic uses, including treatment for or reducing transverse forehead lines, glabellar frown lines, nasolabial fold, vermilion border, periorbital lines, vertical lip lines, oral commissure, etc., as well as defining the lip border. The compositions of the present invention are also useful for correcting contour deformities and distensible acne scars, or for treating other tissue defects, such as, for example, atrophy from disease or trauma or surgically-induced irregularities.

In certain embodiments, the injectable compositions of the present invention are used for surgical procedures involving tissue augmentation, tissue repair, or drug delivery. In some aspects, the compositions are used for tissue augmentation in conditions such as urinary incontinence, vasicoureteral reflux, and gastroesophageal reflux. For example, compositions of the present invention may be used to add tissue bulk to sphincters, such as a gastric or urinary sphincter, to provide proper closure and control. In instances of urinary incontinence, such as stress incontinence in women or incontinence following a prostatectomy in men, the compositions of the invention may be provided to further compress the urethra to assist the sphincter muscle in closing, thus avoiding leakage of urine from the bladder.

Similarly, gastroesophageal reflux disease (GERD, also known as peptic esophagitis and reflux esophagitis) is a disorder that affects the lower esophageal sphincter, the muscle connecting the esophagus with the stomach. GERD occurs when the lower esophageal sphincter is incompetent, weak, or relaxes inappropriately, allowing stomach contents to flow up into the esophagus (i.e., reflux). Malftmction of the lower esophageal sphincter muscles, such as that resulting from muscle tonal loss, can lead to incomplete closure of the lower esophageal sphincter, causing back up of acid and other contents from the stomach into the esophagus. Poor response to dietary modification or medical treatment may require surgery to correct the dysfunction. In one embodiment, collagen compositions of the present invention are used in such procedures and, for example, are injected into the area of the esophageal sphincter to provide bulk to the lower esophageal sphincter.

In other embodiments, the implantable collagen compositions of the invention are used to fill or block voids and lumens within the body. Such voids may include, but are not limited to, various lesions, fissures, diverticulae, cysts, fistulae, aneurysms, or other undesirable voids that may exist within the body; and lumens may include, but are not limited to, arteries, veins, intestines, Fallopian tubes, and trachea. For example, an effective amount of the present composition may be administered into the lumen or void to provide partial or complete closure, or to facilitate repair of damaged tissue.

In other aspects, tissue repair is achieved by providing the collagen composition of the present invention to an area of tissue that has been diseased, wounded, or removed. In some embodiments, compositions of the invention are used to fill in and/or smooth out soft tissue defects such as pockmarks or scars. In such cases, a formulation of the present invention is injected beneath the imperfection. The improved persistence of the present compositions would be beneficial, e.g., by reducing the number and frequency of treatments required to obtain a satisfactorily result. In certain embodiments, the collagen compositions are used for intracordal injections of the larynx, thus changing the shape of this soft tissue mass and facilitating vocal function. Such use is specifically contemplated for the treatment of unilateral vocal cord paralysis. Further, the present invention contemplates use of the collagen compositions in mammary implants, or to correct congenital anomalies, acquired defects, or cosmetic defects.

The present collagen compositions can also be used in various surgical or other procedures for remodeling or restructuring of various external or internal features, e.g., plastic surgery for corrective or cosmetic means, etc.

In any of the embodiments described above, the present compositions may be used for drug delivery, for example, to deliver drugs to an injection site. The drugs can be delivered in a sustained manner from an in vivo depot formed by the collagen upon injection of an implantable composition of the present invention. Drugs delivered in this manner may thus enhance tissue repair, and could provide additional therapeutic benefit.

In additional embodiments, the invention further contemplates incorporation of cells into the implantable collagen compositions to provide a means for delivering cells to repopulate a damaged or diseased tissue or to provide products synthesized by the cells to the tissues surrounding the injection site.

In any of the embodiments described above, the implantable compositions of the present invention may be delivered or administered by any suitable method known or contemplated by those of skill in the art. The invention specifically contemplates delivery by injection, e.g., using a syringe. In some embodiments, the collagen compositions may additionally contain a biocompatible fluid that functions as a lubricant to improve the injectability of the formulation. The collagen compositions of the invention can be introduced into the tissue site by injection, including, e.g., intradermal, subdermal, or subcutaneous injection.

It is understood that the compositions of the present invention can include additional components suitable to the particular formulation. For example, in certain embodiments, the implantable compositions of the present invention are intended for injection and are formulated in aqueous solutions. The compositions can be formulated to include pharmaceutically acceptable carriers and excipients. Such carriers and excipients are well-known in the art and can include, e.g., phosphate buffered saline (PBS) solutions, various solvents, and salts, etc., for example, physiologically compatible buffers including physiological saline buffers such as Hanks's solution and Ringer's solution.

The amount of collagen appropriately included in a particular formulation is determined as standard in the art for such formulations, and is dictated by the intended use. In certain embodiments, the present invention provides implantable compositions comprising collagen wherein the collagen is in aqueous solution at a concentration between about 20 to about 120 mg/ml. In some embodiments, the collagen is in aqueous solution at a concentration between about 30 to about 90 mg/ml; or a concentration of between about 20 to 65 mg/ml; or a concentration of between about 25 to 40 mg/ml. In preferred embodiments, the collagen compositions of the present invention have a collagen concentration of about 35 mg/ml or a collagen concentation of about 65 mg/ml.

### EXAMPLES

The invention will be further understood by reference to the following examples, which are intended to be purely exemplary of the invention. These examples are provided solely to illustrate the claimed invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications are intended to fall within the scope of the appended claims.

### Example 1

### Production of Synthetic Collagen

Synthetic collagens were produced using known methods, as described in, e.g., Vuorela et al. (1997) EMBO J 16:6702-6712 and Nokelainen et al. (2001) Yeast 18:797-806, each of which is incorporated by reference herein in its entirety, with modifications as described below.

Production of other collagens suitable for use in the present compositions can be specifically engineered using molecular biology techniques know to one of skill in the art. Such collagens can be modified by, e.g., an alteration in the polypeptide coding sequence, including deletion, substitutions, insertions, etc., to increase resistance to degradation. For example, synthetic collagens with alterations in the amino acid sequence at specific protease cleavage sites can be produced. An exemplary collagen for use in the present compositions is a synthetic human type III collagen which contains an isoleucine to proline substitution at amino acid residue 1205 of the human type III collagen sequence of SEQ ID NO:1.

Synthetic human type III collagen was prepared essentially as described previously (Vuorela et al. (1997) EMBO J 16:6702-6712). A polynucleotide encoding human α1(III) procollagen was used. Linearized plasmid containing a polynucleotide encoding human α1(III) procollagen was resuspended in deionized(di)H₂O at ˜1 μg/mL and electroporated into a Pichia pastoris strain that expresses the α and β subunits of human prolyl hydroxlase (Vuorela et al. (1997) EMBO J 16:6702-6712). A selected yeast strain was grown in a 100 L fermentation tank in basal salt glycerol medium, pH 5. The strain was grown in the fed-batch mode on glycerol until the wet cell weight reached 215 g/L (6 hours). The glycerol feed was changed to methanol feed for 110 hours, to a final wet cell weight of 380 g/L. Cells were washed with water, lysed in citric acid buffer, and incubated for 48 hours to release synthetic human type III collagen from the cells.

Pepsin was added to the collagen (i.e., procollagen) solution to remove N— and C-propeptide regions from the synthetic collagen. The non-specific nature of pepsin cleavage of collagen resulted in the N— and C-telopeptide regions having variable lengths, as known in the art. The synthetic human type III collagen was then recovered by acid salt precipitation. The precipitated type III collagen was dissolved in 0.1M HCl and clarified by centrifugation and filtration. The collagen solution was then adjusted to pH 5.8 with Tris base in order to precipitate more impurities, then clarified by centrifugation and filtration through a 0.2 μm membrane. The collagen solution was adjusted to pH 9.0 with Tris base and to a final concentration of 1M urea, and then passed over a DEAE-Sepharose FF column (6 L). Synthetic human type III collagen was precipitated from the DEAE flowthrough by addition of HCl and NaCl. The precipitate was concentrated by centrifugation and dissolved in 0.1M HCl. The collagen solution was then clarified by 0.2 μm membrane filtration and diafiltered into 10 mM HCl. The collagen solution was then adjusted to pH 7.2 by adding 1/10 volume of 0.2M Na—PO4, pH 11.2, and incubated overnight to precipitate the collagen. Precipitated synthetic human type III collagen was collected by centrifugation, dissolved in 0.1M HCl, and filtered through a 0.2 μm membrane filter. The filtrate was diafiltered into 10 mM HCl, sterile filtered through a 0.2 μm membrane filter, and stored at 4-8° C.

Synthetic human type I collagen was prepared essentially as described previously (Nokelainen et al. (2001) Yeast 18:797-806). A polynucleotide encoding human α1(I) procollagen and a polynucleotide encoding human α2(I) procollagen was used. A selected yeast strain obtained as described in Nokelainen et al., supra, was grown in a 100 L fermentation tank in basal salt glycerol medium, pH 5. The strain was grown in the fed-batch mode on glycerol until the wet cell weight reached 170 g/L (18 hours). The glycerol feed was changed to methanol feed for 100 hours, to a final wet cell weight of 350 g/L. Cells were washed with water, lysed in citric acid buffer, and incubated for 48 hours to release synthetic human type I collagen from the cells.

Pepsin was added to the collagen (i.e., procollagen) solution to remove N— and C-propeptide regions from the synthetic collagen. The non-specific nature of pepsin cleavage of collagen resulted in the N— and C-telopeptide regions having variable lengths, as known in the art. The synthetic human type I collagen was then recovered by acid salt precipitation. The precipitated type I collagen was dissolved in 0.1M HCl and clarified by centriftigation and filtration. The collagen solution was adjusted to pH 9.0 with Tris base and to a final concentration of 1M urea, and then passed over a DEAE-Sepharose FF column (6 L). Synthetic human type I collagen was precipitated from the DEAE flowthrough by addition of HCl and NaCl. The precipitate was concentrated by TFF and centrifugation and dissolved in 0.1M HCl. The collagen solution was then clarified by 0.2 μm membrane filtration and diafiltered into 10 mM HCl. The synthetic human type I collagen was sterile filtered through a 0.2 μm membrane filter, and stored at 4-8° C.

Collagen fibrils were formed from the synthetic human type III collagen and synthetic human type I collagen as follows. Fibrillogenesis buffer (0.2 M Na₂HPO₄, pH 11.2) was added to a 0.3% (3 mg/ml) solution of synthetic human type III collagen and of synthetic human type I collagen at a 1:10 (v/v) ratio. The solution was incubated at room temperature from 4 hours to overnight. Synthetic human type III collagen fibrils and synthetic human type I collagen fibrils were then collected by centrifugation at 15,000×g for 30 minutes at 10° C.

The pellet was resuspended in PBS to the original volume, and then centrifuged as before. The resultant pellet was transferred to a 10 cc syringe (Becton, Dickinson and Co., Franklin Lakes N.J.) and connected via a Luer Lok-to-Luer Lok connector (Baxa, Englewood Colo.) to a second 10 cc syringe containing ⅕^{th} volume 0.9% NaCl (Irrigation USP; Braun-McGaw, Detroit Mich.). The synthetic collagen was transferred into the second syringe through the connector, air bubbles were removed from the resulting collagen suspension, and then the synthetic collagen was transferred approximately 20 times between the syringes. The connector was then replaced with an 18 G micro-emulsifying needle (Popper Precision Instruments, Lincoln R.I.) and the synthetic collagen was passed through the needle approximately 20 times to further homogenize the material. The mixture was then adjusted to 35 mg/ml collagen and 3 mg/ml lidocaine (lidocaine hydrochloride, USP; Wyckoff Chemical Co, Inc., South Haven Mich.) in 0.9% PBS and the collagen mixture was passed through an 18 G needle as before. Final homogenized synthetic collagen was stored at 4° C.

### Example 2

### Persistence of Synthetic Human Collagen in vitro

The effects of collagenases on synthetic human type III collagen and synthetic human type I collagen, each substantially free of intermolecular and intramolecular crosslinks, were examined. Matrix metalloproteinases (MMPs), also referred to as collagenases, are enzymes capable of cleaving triple-helical collagen. Both MMP-1 (collagenase 1, fibroblast collagenase) and MMP-8 (collagenase 2, neutrophil collagenase) cleave triple-helical collagen types I, II, and III.

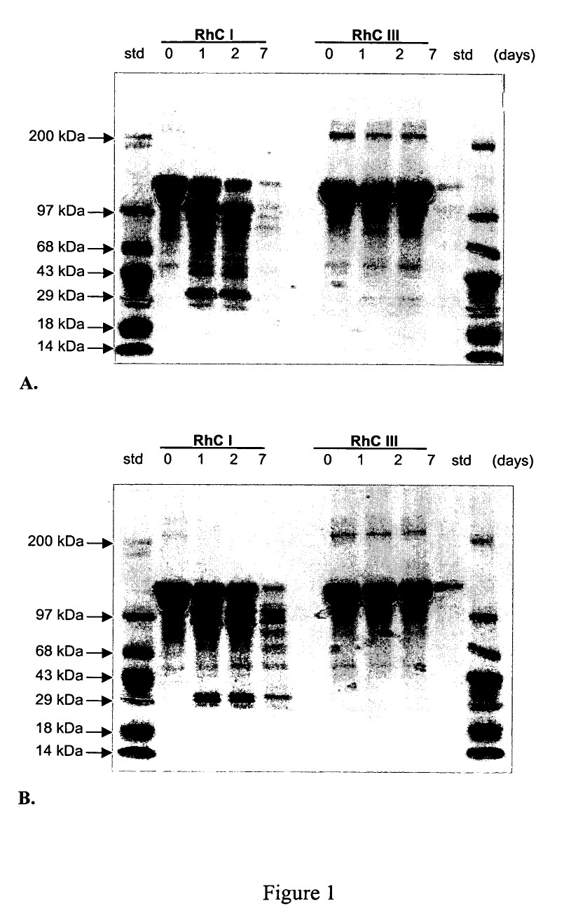

Synthetic human type I collagen and synthetic human type III collagen were prepared as described above in Example 1. MMP-1 pro-enzyme and MMP-8 were obtained from EMB Biosciences (San Diego, Calif.). MMP-1 was converted from the pro-enzyme to the active enzyme according to protocols supplied by the manufacturer. Separate reactions were set up for each MMP used. One milliliter volumes of 2 mg/mL collagen, 50 mM Tris-HCl (pH 7.0), 300 mM NaCl, 5 mM CaCl₂, 0.001 mM ZnCl₂, 0.05% Brij-35, and 0.05% NaN₃ were incubate at 37° C. for one hour. A 200 μL volume was removed from each reaction, and then 0.5 μg MMP-1 or MMP-8 (100-500 mU/mg activity) was added, and the reactions were again incubated at 37° C. Aliquots of 200 μl were removed from each reaction after each of 1, 2, and 7 days incubation. Each 200 μl aliquot was quenched over 20% ethanol/10% trichloroacetic acid and then centrifuged to pellet. The pellets were resuspended in 200 μL 10 mM HCl and fractionated by SDS-PAGE to determine the extent of degradation of the synthetic collagens. The results are shown in FIGS. 1A and 1B.

As can be seen in FIGS. 1A and 1B, both synthetic human type I and type III collagen were cleaved by MMP-1 (FIG. 1A) and MMP-8 (FIG. 1B). However, the rate and extent of MMP degradation of synthetic human type I collagen was substantially higher than that of synthetic human type III collagen for both MMP-1 and MMP-8 enzymes.

For synthetic human type III collagen, the rate of cleavage was slower than that observed for synthetic human type I collagen. Synthetic human type III collagen was degraded by MMP-1 to a greater extent than by MMP-8, which is expected since MMP-1 has a higher specificity for type III collagen. This data showed that collagens substantially free of intermolecular and intramolecular crosslinks demonstrated a certain degree of resistance to degradation by collagenases. These results suggested that collagen substantially free of intermolecular and intramolecular crosslinks can suitably be used in various tissue augmentation procedures. The results further demonstrated that type III collagen is more resistant to degradation by collagenases than is type I collagen, indicating that type III collagen has a higher degree of persistence than does type I collagen.

### Example 3

### Persistence of Synthetic Human Collagen in vivo

In vivo persistence of implanted synthetic human type I collagen and synthetic human type III collagen was investigated as follows. Wistar rats (Charles River Laboratories, Inc.) were shaved and an 8 cm×6 cm site for injection was marked the day prior to implantation. Purified collagen preparations in 10 mM HCl at a concentration of 3 mg/mL were mixed with 1/10^{th} volume of 0.2 M NaPO₄, pH 11. The collagen fibrils were collected by centrifugation at 10,000×g for 15 minutes at 4° C., resuspended in PBS at a collagen concentration of 35 mg/ml, and lidocaine was added to a final concentration of 3 mg/ml.

Implants were made by subcutaneous injection of 0.5 ml of a 35 mg/ml suspension of synthetic human type I collagen, synthetic human type III collagen, or bovine type I collagen (VITROGEN, Cohesion Technologies) in PBS on the dorsal flank. The collagen suspension was injected using a 1 cc syringe with a 28-gauge needle. Each animal received three separate injections of collagen material. For each collagen material tested, a total of 9 implants (3 rats, 3 implants/rat) were present at the start of the study. Groups of three animals per test material were analyzed at the following time points post implantation: day 2, and week 4, 8, 13, 26, 39, and 52 post implantation. Implants were surgically removed and dissected free from surrounding tissue, and examined macroscopically for appearance and texture. Essentially no inflammatory or tissue response was observed following implantation of either synthetic human collagen.

As is standard in the art, the persistence of the collagen implants was evaluated by determining the number of the original implants that were present at the injection sites at each of the time points. The data are summarized in Table 1 below and are presented as the number of the total implants remaining/number of the original implants at each time point. Surprisingly, all original implants containing type I collagen substantially free of intramolecular and intermolecular crosslinks and all original implants containing type III collagen substantially free of intramolecular and intermolecular crosslinks were present through week 13. Also surprisingly, the implants containing type III collagen showed persistence markedly greater than those of the implants containing type I collagen, and more than half of these implants remained at 52 weeks.

TABLE 1

| | Synthetic human | Synthetic human |
|---|---|---|
| Time Point | type I collagen | type III collagen |
| Day 2 | 9/9 | 9/9 |
| Week 4 | 9/9 | 9/9 |
| Week 8 | 9/9 | 9/9 |
| Week 13 | 9/9 | 9/9 |
| Week 26 | 3/9 | 6/9 |
| Week 39 | 1/9 | 5/9 |
| Week 52 | 1/9 | 5/9 |

These results showed that, first, collagen substantially free of intermolecular and intramolecular crosslinks demonstrated unexpected persistence upon implantation, and is thus suitable for use in various tissue augmentation applications. Additionally, these results demonstrate that type III collagen was more persistent than type I collagen, and that compositions containing exclusively type III collagen, or collagen that is predominantly type III collagen, can provide unexpected benefits, e.g., enhanced persistence.

Various modifications of the invention, in addition to those shown and described herein, will become apparent to those skilled in the art from the foregoing description. Such modifications are intended to fall within the scope of the appended claims.

All references cited herein are hereby incorporated herein by reference in their entirety.

What is claimed is:

1. An implantable composition comprising collagen, wherein the collagen consists of type III collagen.

2. The implantable composition of claim 1, wherein the type III collagen is human type III collagen.

3. The implantable composition of claim 1, wherein the type III collagen is synthetic type III collagen.

4. The implantable composition of claim 1, wherein the implantable composition is selected from the group consisting of a dermal filler, a bone-void filler, and a dental implant.

5. The implantable composition of claim 1, wherein the implantable composition is suitable for use in tissue augmentation.

6. The implantable composition of claim 5, wherein the tissue augmentation is soft tissue augmentation.

7. The implantable composition of claim 5, wherein the tissue augmentation is hard tissue augmentation.

8. An implantable composition comprising collagen, wherein the collagen comprises at least 60% type III collagen.

9. An implantable composition comprising collagen, wherein the collagen is substantially free of intermolecular and intramolecular crosslinks.

10. The implantable composition of claim 9, wherein the collagen is a synthetic collagen.

11. The implantable composition of claim 9, wherein the collagen is a fibrillar collagen.

12. The implantable composition of claim 9, wherein the collagen is selected from the group consisting of type I collagen, type II collagen, and type III collagen.

13. An implantable composition comprising collagen, wherein the collagen is substantially free of endogenous crosslinks.

14. A method for augmenting soft tissue in a subject, the method comprising administering to the subject at an augmentation site a composition comprising collagen, wherein the collagen consists of type III collagen, thereby augmenting the soft tissue.

15. The method of claim 14, wherein the subject is a human.

16. The method of claim 14, wherein the administering is by injection.

17. The method of claim 14, wherein the administering is selected from the group consisting of subcutaneous administration, intradermal administration, and subdermal administration.

18. A method for augmenting soft tissue in a subject, the method comprising administering to the subject at an augmentation site a composition comprising collagen, wherein the collagen comprises at least 60% type III collagen, thereby augmenting the soft tissue.

19. A method for augmenting soft tissue in a subject, the method comprising administering to the subject at an augmentation site a composition comprising collagen, wherein the collagen is substantially free of intermolecular and intramolecular crosslinks, thereby augmenting the soft tissue.

20. A method for preparing an implantable collagen composition, the method comprising providing a collagen substantially free of intermolecular and intramolecular crosslinks, and crosslinking the collagen, thereby preparing an implantable collagen composition.

21. A method for preparing an implantable collagen composition, the method comprising providing a collagen consisting of type III collagen, and preparing an aqueous solution of the collagen, thereby preparing an implantable collagen composition.

22. A method for preparing an implantable collagen composition, the method comprising providing a collagen consisting of at least 60% type III collagen, and preparing an aqueous solution of the collagen, thereby preparing an implantable collagen composition.

23. A method for preparing an implantable collagen composition, the method comprising providing a collagen substantially free of intermolecular and intramolecular crosslinks, and preparing an aqueous solution of the collagen, thereby preparing an implantable collagen composition.

24. A method for preparing an implantable collagen composition, the method comprising providing a collagen substantially free of intermolecular and intramolecular crosslinks, crosslinking the collagen, and preparing an aqueous solution of the crosslinked collagen, thereby preparing an implantable collagen composition.

25. A kit useful for augmenting soft tissue, the kit comprising a collagen consisting of type III collagen, a syringe, and a needle.
